(19) United States
(12) Patent Application Publication
(10) Pub. No.: US 2013/0158459 A1
(43) Pub. Date: Jun. 20, 2013

(54) DEVICE FOR SETTING UP A DILUTION MEASUREMENT SITE

(71) Applicant: PULSION MEDICAL SYSTEMS SE, Feldkirchen (DE)

(72) Inventors: Oliver GOEDJE, Gruenwald (DE); Thomas THALMEIER, Munich (DE); Matthias BOHN, Munich (DE)

(73) Assignee: PULSION MEDICAL SYSTEMS SE, Feldkirchen (DE)

(21) Appl. No.: 13/764,967

(22) Filed: Feb. 12, 2013

Related U.S. Application Data
(62) Division of application No. 11/699,608, filed on Jan. 30, 2007.

(30) Foreign Application Priority Data
Jan. 30, 2006 (EP) 06101019.5

(51) Int. Cl. A61M 1/30

(52) U.S. Cl. CPC A61M 1/30
USPC 604/5.04

(57) ABSTRACT

A device sets up a dilution measurement site on a hemodialyzer for determining the filling status of a dialysis patient during dialysis treatment, by dilution measurements. The arterial connection piece is connected with an arterial fistula needle, and the venous connection piece is connected with a venous fistula needle. The venous connection piece has an injection channel through which a bolus required to perform a dilution measurement can be injected into the blood, which flows through the venous connection piece coming from the hemodialyzer, in the direction of the venous blood vessel access. The arterial connection piece has a sensor measuring the temperature of the blood flowing through the venous connection piece coming from the arterial blood vessel access, in the direction of the hemodialyzer.

### CROSS REFERENCE TO RELATED APPLICATIONS

This application is a divisional of and Applicant claims priority under 35 U.S.C. §§120 and 121 of parent U.S. patent application Ser. No. 11/699,608 filed Jan. 30, 2007, which application is based upon and claims the benefit of priority under 35 U.S.C. §119 of European Patent Application No. 06101019.5, filed on Jan. 30, 2006, the disclosures of each of which are incorporated herein in their entirety by reference. A certified copy of priority European Patent Application No. 06101019.5 is contained in parent U.S. application Ser. No. 11/699,608.

### BACKGROUND OF THE INVENTION

1. Field of the Invention

The present invention relates to a device for setting up a dilution measurement site on a hemodialyzer.

2. Description of the Related Art

In Germany alone, about 50,000 patients have to undergo hemodialysis (blood cleansing treatment) on a regular basis, i.e. several times a week. In this connection, as is sufficiently known, extracorporeal hemodialyzers (so-called “artificial kidneys”) are directly connected to the bloodstream of the patient in question. The connection takes place, in most cases, by way of fistula needles at an arteriovenous fistula, i.e. a surgically created blood vessel connection between an artery and a vein, or by way of a so-called shunt, i.e. an extracorporeal artificial connection line that is connected with an artery and with a vein. In this connection, blood is passed to the hemodialyzer from the arterial side, and passed back into the body on the venous side. Hemodialyzers have not only pumps, heat exchangers, and an air trap that prevents air bubbles from entering the bloodstream on the venous side, but also the dialyzer as the core piece. The latter possesses large membrane surfaces, configured as a capillary system or film system, for example, over which patient blood flows on one side, and a dialysis fluid flows, on the other side. On the basis of diffusive exchange through the membrane surfaces, electrolytes that are at too high a concentration and substances that must be excreted in the urine are removed from the blood. Furthermore, a water exchange takes place, and also, glucose and electrolytes at low concentration can be added, if necessary.

Hemodialyzers are equipped with complicated balancing systems, which, among other things, are supposed to prevent too much fluid from being removed or added to the body of the connected patient by way of water content changes of the dialyzed blood. It is decisive for the patient's well-being and health that his/her bloodstream filling status, i.e. the total circulating blood volume of his/her body, does not go above a reference range and, in particular, does not go below it. For this purpose, in addition to the use of the stated balancing system, the patients are precisely weighed before and after the dialysis treatment, in each instance. Nevertheless, precise balancing is difficult, since dialysis patients frequently take in solid or liquid nutrients during the dialysis treatment, which extends over several hours, and excretions in the form of perspiration, urine, and feces can occur.

### SUMMARY OF THE INVENTION

It is the task of the present invention to create an improvement in this regard, i.e. to allow an assessment of the bloodstream filling level of the dialysis patient that puts little stress on the dialysis patient as well as on the medical personnel in question, is easy to handle, is functionally reliable and convenient.

This task is accomplished, according to one aspect of the present invention, by means of a device for setting up a dilution measurement site on a hemodialyzer. With such a measurement site, the possibility is given of determining the bloodstream filling status of a dialysis patient, by means of dilution measurements, at any desired points in time during the dialysis treatment but in particular, immediately after the treatment starts and immediately before it ends. Checking the fluid balance by means of weighing the dialysis patient, which is subject to error, can be eliminated. Setting up a dilution measurement site by means of a device according to the invention not only allows determining the filling level, but also determining other hemodynamic parameters by means of known thermodilution or indicator dilution techniques. This results in the advantages of simple, reliable, and convenient determination of relevant data, particularly for the monitoring of intensive-care patients whose life depends on hemodialysis.

A method for determining the bloodstream filling level of a patient by means of thermodilution measurements, for the implementation of which a measurement site set up according to the invention can be used in particularly advantageous manner, is described in the German Offenlegungsschrift DE 42 14 402 A1. A method for determining the circulating blood volume of a patient by means of indicator dilution measurements, for the implementation of which a measurement site set up according to the invention can also be used, is known from the German Offenlegungsschrift DE 41 30 931 A1. A measurement site set up according to the invention can also be used for an indicator dilution measurement method in accordance with or similar to the U.S. Pat. No. 6,757,554 B2, or for a combined indicator dilution and thermodilution measurement method (dual indicator dilution technique) as described in the German Offenlegungsschrift DE 101 43 995 A1.

In particular, these and other objects are accomplished by means of a device for setting up a dilution measurement site on a hemodialyzer according to the invention. Particularly advantageous embodiments are discussed below.

As explained above, the device according to the invention is suitable not only for advantageous use of the thermodilution technique but also of the indicator dilution technique, or a combination thereof.

However, most preferably the present invention is implemented for performing (transpulmonary) thermodilution technique. Applying transpulmonary thermodilution technique makes it possible to determine, using algorithms essentially known per se from the prior art, among other parameters, extravascular lung water, which is an important physiological parameter especially in connection with monitoring critically ill patients.

It is thus particularly preferred to implement the present invention in such a manner that cardiovascular parameters can be derived from a thermodilution curve measured using a device configured as described herein. In particular, an especially advantageous embodiment of a system according to the present invention uses temperature readings, from a temperature sensor the arterial connection piece is equipped with, to determine cardiovascular parameters by algorithms known per se in the field of transpulmonary thermodilution. If the present invention is implemented for performing transpulmonary thermodilution technique, it is particularly advantageous to equip the arterial side with a temperature sensor which, in use, is in close thermal contact with the blood to be dialyzed, in order to achieve good accuracy of measurement. Usually, close thermal contact with the blood to be dialyzed in this sense will not be achieved by measuring the blood temperature across the wall of regular tubing such that a relatively thick wall can effect the measurement both as a considerable thermal resistance and a local heat sink thus delaying sensor response. It is therefore preferred to integrate the temperature sensor into the arterial connection piece or to configure the arterial connection piece in such a manner that a suitable temperature sensor device, such as a temperature sensor probe, can be inserted to be in close thermal contact with blood to be dialyzed. Previously known “clamp-on” sensors occasionally used to measure the temperature of liquids, such as blood, flowing through tubes will, in most cases, not be considered to deliver sufficient accuracy of measurement.

The sensor provided in the arterial connection piece can advantageously be configured like known sensors used in dilution measurement methods. A platinum resistor sensor, in particular, is suitable for the temperature measurement; however, other thermoresistors or thermoelements are also practical.

The arterial and the venous connection piece can advantageously be mechanically connected and thereby integrated into a common functional unit, but it is also advantageously possible to implement a separate configuration of the connection pieces. In this connection, the integration of the arterial and the venous connection piece into a shunt is also possible, as is connecting the arterial and the venous connection piece with fistula needles. This connection can advantageously be implemented both in fixed manner, and in releasable manner, for example by means of Luer lock connections. Luer lock connections can also advantageously be provided for the connection to the hemodialyzer at the hemodialyzer input connector and the hemodialyzer output connector, which is generally releasable, but connections configured in another manner, such as screw connections, bayonet connections, catch engagement connections, clamping connections, etc., can also be provided.

The arterial blood vessel connection, in particular (but in principle, also the venous blood vessel connection) can also be implemented by means of a catheter. In this connection, there is the possibility of advantageously integrating the sensor for measuring the temperature of the blood to be dialyzed and/or the sensor for measuring the indicator concentration in the blood to be dialyzed into the catheter, or introducing it through the catheter by means of a probe.

Mechanical and/or electrical, i.e. electronic connection codings can be provided for one or both connections to the hemodialyzer, as well as for other connections, particularly with evaluation hardware to be used, which codings ensure that compatible devices are used, and that connectors of the arterial and the venous side are not interchanged. Furthermore, suitable connection codings can be queried by the hemodialyzer as well as by connected evaluation hardware, in order to adapt operating, service, correction, or calculation parameters to the type of the device according to the invention being used. It is advantageous that corresponding codings can be implemented, for example, as resistors, impedance bridges, electrically connected or transponder-coupled chips, specific pin arrangements, or the like.

The injection channel of the venous connection piece of the device according to the invention can advantageously have equipment characteristics of the injection channel known from the reference EP 1 034 737 A1, but alternatively can also be structured in simpler manner. While this channel is particularly optimized for use of an injectate at room temperature, cooled or heated injectates can also be used alternatively, according to the invention.

According to other aspects, the task is accomplished by means of a dialyzer according to the invention as well as a system according to the invention. Particularly advantageous embodiments are discussed below.

Preferably, in this connection, the program technology device of the evaluation unit is configured for carrying out evaluation steps of a dilution method according to one of the references listed above.

A determination of the injection time point and the injection duration can advantageously be provided, as described in the German Offenlegungsschrift DE 197 38 942 A1. However, this is not absolutely necessary.

According to a particularly advantageous further development of the present invention, a control circuit is provided, which brings about an effect on fluid withdrawal from or fluid enrichment of the blood in the hemodialyzer, as a function of changes in the bloodstream filling level that are determined.

According to yet another aspect of the present invention, the underlying object is accomplished by a method of setting up a dilution measurement site on a hemodialyzer according to the invention, a particularly advantageous embodiment of which can be carried out as discussed below.

According to yet another aspect of the present invention, the underlying object is accomplished by a method of carrying out thermodilution measurements on the cardiovascular system of a dialysis patient according to the invention, a particularly advantageous embodiment of which can be carried out as discussed below.

Generally, any variant of the invention described or indicated within the framework of the present application can be particularly advantageous, depending on the economic and technical conditions in an individual case. Unless something is said to the contrary, and to the extent that this can fundamentally be technically implemented, individual characteristics of the embodiments described can be interchanged or combined with one another.

### BRIEF DESCRIPTION OF THE DRAWINGS

In the following, examples of preferred embodiments of the present invention will be explained in greater detail, using the related drawings.

In this connection, the drawings are purely schematic, and are not representations to scale, for reasons of better illustration. In particular, relationships between the dimensions, particularly of the channel diameter, hose lengths, and outside dimensions, can deviate from actual embodiments. In practice, the dimensions can be dimensioned on the basis of the requirements in an individual case, and on the basis of available standard parts, such as blood vessel access points of conventional hemodialyzers and injection channels that are available on the market.

Elements that correspond to one another in the individual figures are provided with the same reference symbol, to the extent that this makes sense.

FIG. 1 shows a sectional view of a device according to the invention, in which the arterial and the venous connection piece are configured separately and already connected with a fistula needle as the blood vessel access point, in each instance.

FIG. 2 shows a sectional view of a device according to the invention, in which the arterial and the venous connection piece are integrated into a common functional unit.

FIG. 3 shows a system according to the invention, having the device from FIG. 2, shown only in stylized form, a hemodialyzer, and an evaluation unit, whereby the device is connected to the bloodstream of a dialysis patient by way of two fistula needles.

FIG. 4a shows a device according to the invention similar to FIG. 2, whereby, however, a catheter is provided for the arterial blood vessel connection, and the temperature sensor has been moved into the catheter. The catheter is shown in interrupted and non-sectioned form.

FIG. 4b-c illustrate two different variants of the temperature sensor arrangement, using a sectional representation of the distal end of the catheter that is enlarged as compared with FIG. 4a.

### DETAILED DESCRIPTION OF PREFERRED EMBODIMENTS

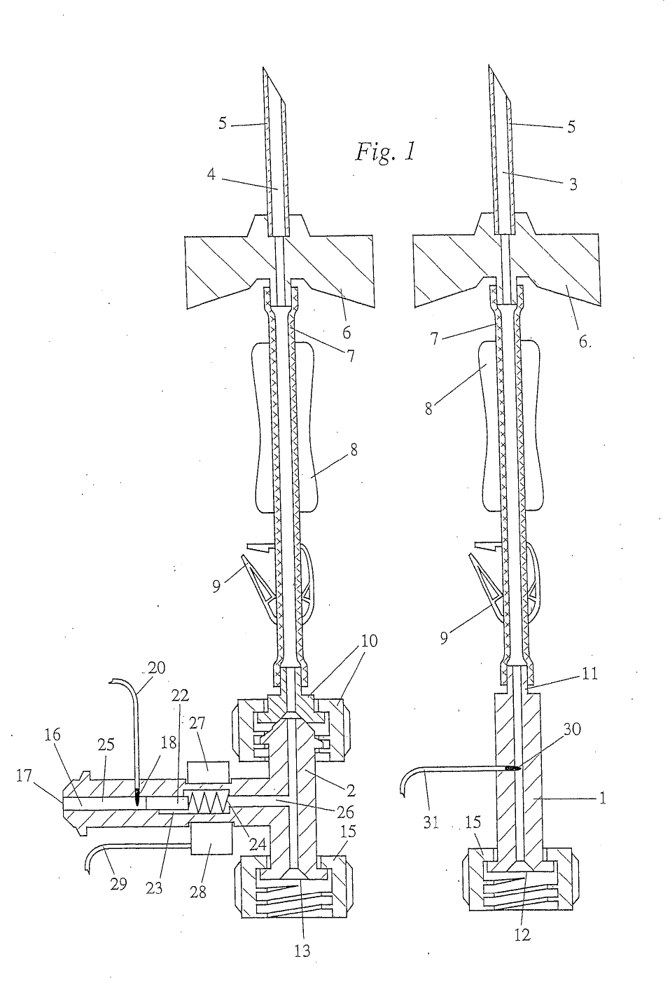

FIG. 1 shows a device according to the invention, the arterial connection piece 1 and venous connection piece 2 of which are structured as separate components. The arterial connection piece 1 is connected with an arterial fistula needle 3, and the venous connection piece 2 is connected with a venous fistula needle 4, as the blood vessel access. The fistula needles 3, 4 have a ground hollow needle 5, a plastic handle piece 6, as well as a hose piece 7 with a hose sleeve 8 and a squeeze clamp 9, in each instance. The plastic handle piece 6 is preferably marked with a color, for example red for the arterial and blue for the venous blood vessel access.

The connection between connection pieces 1, 2 and fistula needles 3, 4 can be structured by way of a simple hose tap connection 11, for example, as shown for the arterial connection piece 1, or also in some other manner, for example by way of a Luer lock connection 10 or another quick connection, as shown for the venous connection piece 2. The hemodialyzer input connector 12 on the arterial connection piece 1 and the hemodialyzer output connector 13 on the arterial connection piece 2 are preferably also structured in such a manner that a quick connection can be produced with the connectors of a hemodialyzer 14, for example by way of Luer lock nuts 15.

The venous connection piece 2 has an injection channel 16, through which a bolus required to carry out a dilution measurement can be injected into the blood, which flows through the venous connection piece 2 coming from the hemodialyzer output connector 13, in the direction of the venous blood vessel access. Not shown is a valve that can optionally be provided in the region of the hemodialyzer output connector 13, in order to prevent injectate from getting through the hemodialyzer output connector 13 in the direction of the hemodialyzer when the hemodialyzer is shut off (i.e. when the bloodstream 39 through the hemodialyzer 14 has come to a stop). This can be a simple kick-back valve, but alternatively also a (semi)-automatic or manually activated valve, for example a solenoid valve electronically controlled by a connected hemodialyzer 14, which closes when the hemodialyzer 14 is shut off.

The injection channel 16 shown has a connector 17 to which a syringe, injection pump, or other device for supplying injectate can be connected. An injection pump can advantageously be controlled by way of a device that also serves as an evaluation unit 21 for the injection measurements.

Furthermore, the injection channel 16 is equipped with a temperature sensor 18 for determining the injectate temperature. The tip, projecting into the injection channel 16, of a platinum thermoresistor sensor having a signal line 20 (merely indicated) that runs in a cable (possibly shielded), and can be connected to an evaluation unit 21 by way of a plug (possibly mechanically and/or electrically or electronically coded) is shown merely schematically. Furthermore, the injection channel 16 has a valve that opens when a minimum injection pressure is exceeded and, at the same time, exercises a switching function by way of which the injection time point and/or the injection duration is/are determined.

The valve consists essentially of a cylinder 22 guided in the interior of the injection channel 16, one or more longitudinal groove(s) 23 in the interior of the injection channel 16, which extend over only part of the cylinder length, and a pressure spring 24, which presses the cylinder 22 counter to the injection direction. Preferably, a stop (not shown) is provided, against which the cylinder 22 rests if no injection pressure or only a slight injection pressure prevails in the injection channel 16. The pressure spring 24 is then preferably slightly biased. If the injection pressure increases, the spring 24 is compressed. When a threshold value is exceeded, the cylinder lift is great enough so that the longitudinal groove 23 produces a continuous connection between the proximal 25 and the distal 26 part of the injection channel 16 for the injectate, and the injectate thus can enter into the blood flowing from the hemodialyzer output connector 13 in the direction of the venous blood vessel access. At the end of the injection, the re-set force of the spring 24 presses the piston 22 back into its starting position.

In order to exert the switching function for determining the injection time point and/or the injection duration (preferably both), the cylinder 22 consists of ferromagnetic metal and acts together with a magnet 27 and a reed switch 28. If the cylinder 22 is situated between magnet 27 and reed switch 28 (during the injection), the magnetic field is deflected. If, on the other hand, the cylinder 22 is not situated between magnet 27 and reed switch 28 (before and after the injection), the reed switch 28 is activated by means of the effect of the magnetic field. Querying of the reed switch 28 by way of a signal line 29 (only indicated in the drawing) allows the determination of the injection time point and/or injection duration (preferably of both). The signal line 29 can run (possibly together with the signal line 20 of the temperature sensor 18) in a cable (possibly shielded), and can be connected to the evaluation unit 21 by way of a plug (possibly mechanically and/or electrically or electronically coded) (not shown).

The valve or the switch, respectively, can, of course, also be structured differently from what was presented above. For example, the time point and the duration of the injection can also be determined by way of a capacitative measurement or by way of the temperature measurement. Furthermore, the injection time point can be manually recorded, e.g. by way of a button that is operated by the personnel performing the injection. Depending on the demands on the quality of the measurement technology, a simpler embodiment of the injection channel 16, without a valve or switch and/or without a sensor, is also possible. An injection channel 16 that can be heated can also be implemented.

The arterial connection piece 1 has a temperature sensor 30 by means of which the temperature of the blood, which flows through the venous connection piece 2, coming from the arterial blood vessel access, in the direction of the hemodialyzer input connector 12, can be measured. By way of this temperature measurement, the system response to the disruption caused in the circulatory system of the dialysis patient 32 by means of the bolus injection can be determined.

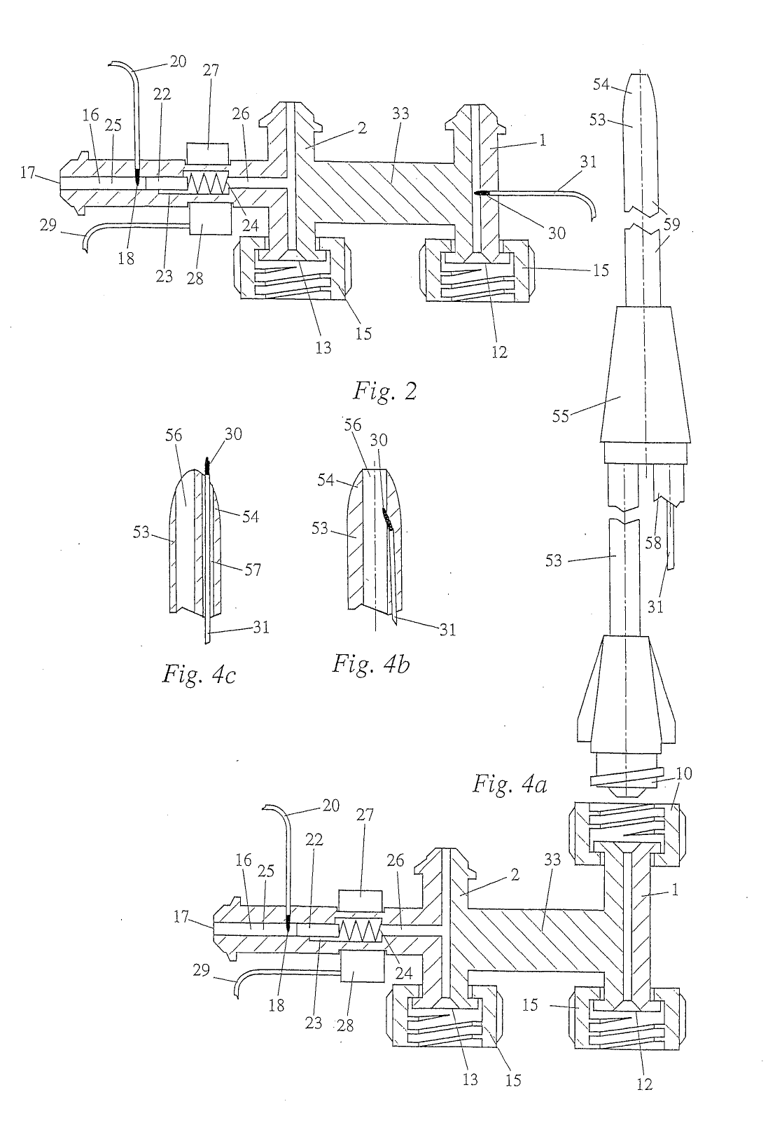

The tip, projecting into the interior of the arterial connection piece 1, of a platinum thermoresistor sensor having a signal line 31 (merely indicated) that runs in a cable (possibly shielded), and can be connected to the evaluation unit 21 by way of a plug (possibly mechanically and/or electrically or electronically coded) (not shown) is shown merely schematically. In the case of integration of arterial and venous connection piece (1, 2) into a common functional unit, as shown in FIG. 2, all of the signal lines 20, 29, 31 can also run in a common cable.

Preferably, the connection pieces 1, 2 are structured as disposable products, for reasons of hygiene, and are packaged in sterile packaging, individually or together, with or without fistula needles 3, 4.

The structure and method of functioning of the device according to the invention shown in FIG. 2 are essentially the same as explained in connection with FIG. 1. However, arterial and venous connection piece (1, 2) are connected with one another as a common functional unit 33. Quick connections for connecting fistula needles (3, 4) are provided for both blood vessel accesses.

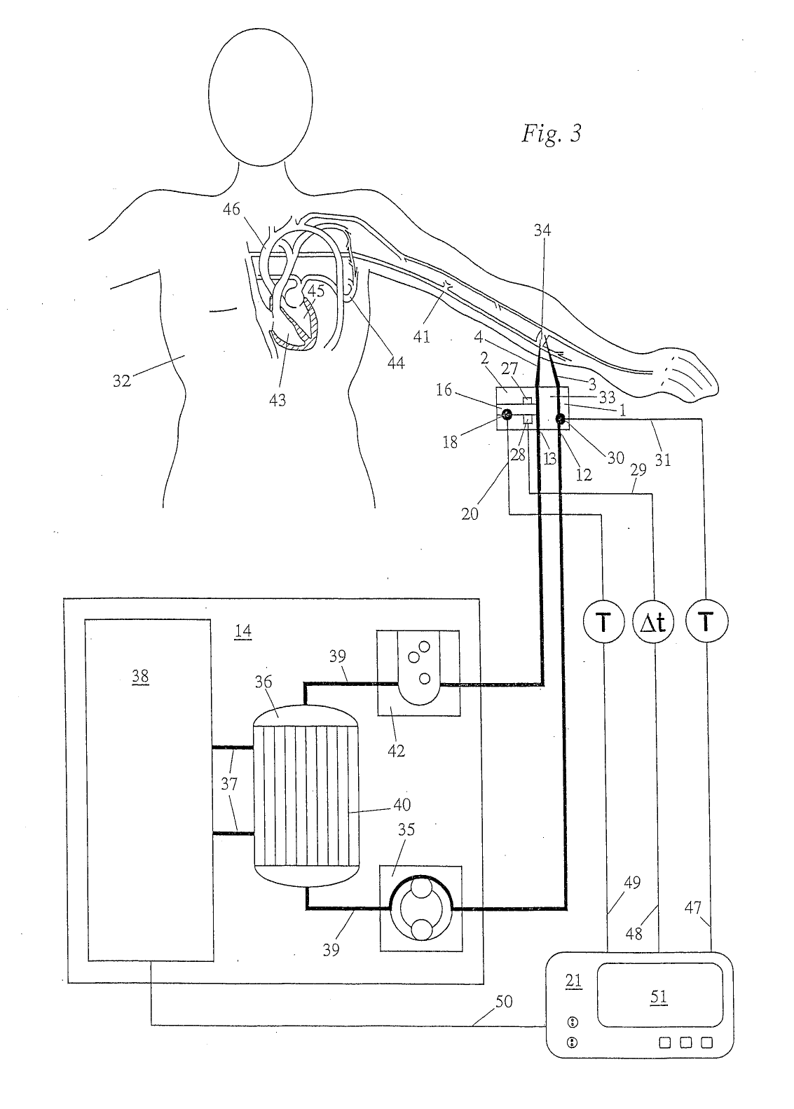

FIG. 3 shows a system according to the invention, in which a device 33 according to FIG. 2 or having a similar structure is connected with a hemodialyzer 14. The signal lines 20, 29, and 31 are connected with the evaluation unit 21, which is equipped in terms of program technology for evaluating the thermodilution measurements, which can be carried out using the device 33 according to the invention.

An arterial and a venous blood vessel access are produced at an arteriovenous fistula 34 of a dialysis patient 32, by means of fistula needles (3, 4), which are connected with the arterial and venous connection piece (1, 2), respectively, in each instance.

Blood to be dialyzed flows from the arterial blood vessel access, transported by a blood pump 35, through the arterial connection piece 1, towards the hemodialyzer 14. As a rule, the blood is heparinized. In the actual dialyzer 36, which is equipped with a large membrane surface 40, transmembranous substance exchange with the permeate, and thus “blood washing,” takes place. The permeate stream 37 runs through a complicated balancing system 38, now shown in greater detail here, which can fundamentally be implemented as in conventional hemodialyzers. The electrolyte and fluid content of the permeate are precisely adjusted in the balancing system 38, furthermore the permeate is ultra-filtered, tempered by means of heat exchangers, de-gassed, and examined for leakage blood.

After the bloodstream 39 flows over the membrane surface 40 in the actual dialyzer 36, it is passed through an air trap 42 and then enters into the venous connection piece 2 through the hemodialyzer output connector 13. From there, the blood gets back into the bloodstream 41 of the patient 32 through the venous blood vessel access.

In the venous connection piece 2, a bolus can be injected by way of the injection channel 16; the temperature of this bolus differs from the blood temperature. Preferably, the temperature, time point, and if applicable also the duration of the bolus injection are measured in the manner described above, using the temperature sensor 18 and the reed switch 28, and the measurement values are passed to the evaluation unit 21.

The local temperature change imposed on the patient's blood in such a manner continues with the flow direction of the blood, and reaches the right atrium, the right ventricle 43, the pulmonary circulation 44, the left atrium, the left ventricle 45 and, by way of the aorta 46, the bloodstream 41 of the patient 32 again, one after the other. In this way, the system response to the disruption caused by the bolus injection can be recorded by means of the temperature sensor 30 in the arterial connection piece 1. The thermodilution curve results from the temperature progression (i.e. the progression of the temperature deviation from the normal blood temperature) over time.

Based on known approaches of transpulmonary thermodilution techniques, the evaluation unit 21 can therefore calculate various hemodynamic parameters, particularly, however, the global end-diastolic volume GEDV and thus the filling status of the patient 32. For this purpose, the evaluation unit 21 receives the arterial temperature measurement data from the temperature sensor 30, by way of the input channels 47, 48, 49, as well as the measurement variables of injectate temperature, injection duration, and injection time point, which characterize the bolus injection.

The global end-diastolic volume GEDV can be determined according to the equation

GEDV=CO·(MTT−DST)

In this equation, CO is the cardiac output, MTT is the mean transit time, and DST is the exponential downslope time, i.e. the time that the temperature deviation attributable to the bolus injection requires to drop by a factor of 1/e.

The cardiac output CO can be determined by means of algorithms that are based on the Stewart-Hamilton equation:

$$C O = \frac{{V}_{L} ( {T}_{B} - {T}_{L} ) {K}_{1} {K}_{2}}{\int Δ {T}_{B} ( t ) d t}$$

In this equation, T_{B} is the initial blood temperature, T_{L} is the temperature of the bolus used as the thermoindicator, in other words the measured injectate temperature, V_{L} is the volume of the injected bolus, and ΔT_{B}(t) is the deviation of the blood temperature from the base-line temperature T_{B} as a function of the time t. K₁ and K₂ are constants to be determined empirically or estimated, to take the specific measurement arrangement into consideration.

The hemodynamic parameters determined can be output by way of the display 52 that also serves to guide the operator. Furthermore, the evaluation unit 21 can also be equipped with an external memory medium or with a printer.

Furthermore, the balancing system 38 can be controlled by way of the output channel 51. In the case of excessive deviations of the global end-diastolic volume GEDV from a patient-specific reference value, a correction of the filling status of the patient 32 can thus be achieved by way of targeted raising or lowering of the fluid content. A corresponding control is optional. Alternatively, the treating physician can take appropriate counter-measures on the basis of the output calculated GEDV value.

An alternative embodiment of the arterial side is illustrated in two different variants, using FIG. 4a-c. Here, a catheter 53 is provided for the arterial blood vessel access, which is shown non-sectioned, separated from the arterial connection piece 1, and interrupted, for reasons of space, in FIG. 4a. In FIGS. 4b and 4c, two different variants of the arrangement of the temperature sensor 30 are illustrated, on the basis of a sectional representation of the distal catheter end 54, which is enlarged as compared with FIG. 4a, in each instance. The venous side is configured as in FIG. 2.

The catheter 53 can be connected with the arterial connection piece 1 by way of a Luer lock connection 10 or a similar quick connection at its proximal end. In addition, it can also be advantageous to connect catheter 53 and arterial connection piece 1 permanently with one another, as a common part, for example by means of bonding or gluing.

From the proximal end of the catheter 53, a blood guide lumen runs to the distal catheter end 54. Arterial blood to be dialyzed is passed to the arterial connection piece 1 by means of the blood guide lumen 56.

Distal to the channel separation 55, the signal lines 31 of the temperature sensor 30 and the blood guide lumen 56 run in a common catheter body 59. The temperature sensor 30 can be rigidly integrated into the catheter 53, as shown in FIG. 4b, or it can be structured as part of a separate probe, which is introduced through a probe lumen 57, as shown in FIG. 4c. According to another variant, not shown, the temperature sensor 30 can also be structured as a separate probe, which is guided in the blood guide lumen 56.

Measuring the temperature of the blood to be dialyzed can take place either in free blood flow, as shown in FIG. 4c, in that the temperature sensor 30 projects beyond the distal catheter end 54, or in the interior of the blood guide lumen 56, as shown in FIG. 4b.

Proximal to the channel separation 55, the signal line 31 runs in a separate hose piece or cable 58, and can be connected with the evaluation unit 21 by way of a plug (possibly mechanically and/or electrically or electronically coded) (not shown).

What is claimed is:

1. Method of setting up a dilution measurement site on a hemodialyzer, said method including the steps of
connecting an arterial connection piece to a hemodialyzer input,
connecting said arterial connection piece to an arterial blood vessel access,
installing at least one of a sensor for measuring the temperature of blood to be dialyzed and a sensor for measuring an indicator concentration in blood to be dialyzed,
connecting a venous connection piece, having an injection channel for injecting a bolus into dialyzed blood that flows through the venous connection piece, to a hemodialyzer output, and
connecting said venous connection piece to a venous blood vessel access.

2. Method according to claim 1, wherein, for said arterial connection piece, an arterial connection piece is used into which at least one of said sensor for measuring the temperature of blood to be dialyzed and said sensor for measuring an indicator concentration in blood to be dialyzed is integrated.

3. Method of carrying out thermodilution measurements on the cardiovascular system of a dialysis patient, said method including the steps of
setting up a dilution measurement site on a hemodialyzer by connecting an arterial connection piece to a hemodialyzer input, connecting said arterial connection piece to an arterial blood vessel access, installing a sensor for measuring the temperature of blood to be dialyzed, connecting a venous connection piece, having an injection channel for injecting a bolus into dialyzed blood that flows through the venous connection piece, to a hemodialyzer output, and connecting said venous connection piece to a venous blood vessel access,
injecting said bolus into said dialyzed blood through said injection channel, wherein said bolus has a temperature different from said dialyzed blood,
acquiring at least one variable characterizing said bolus injection, said at least one variable including at least one of temperature of said bolus injected, amount of said bolus injected and duration of said injection of said bolus,
measuring, using said sensor, the temperature of the blood to be dialyzed over time,
evaluating a transpulmonary diffusion measurement, in that at least one hemodynamic parameter is determined proceeding from the measured temperature of the blood to be dialyzed over time and the at least one variable characterizing said bolus injection.

4. Method according to claim 3, wherein, for said arterial connection piece, an arterial connection piece is used into which said sensor for measuring the temperature of the blood to be dialyzed is integrated.
